(19) United States
(12) Patent Application Publication
(10) Pub. No.: US 2023/0355448 A1
(43) Pub. Date: Nov. 9, 2023

(54) Feminine Hygiene Assembly

(71) Applicants: Tony Griffith, Solon, OH (US); Brittany Griffith, Solon, OH (US)

(72) Inventors: Tony Griffith, Solon, OH (US); Brittany Griffith, Solon, OH (US)

(21) Appl. No.: 17/737,402

(22) Filed: May 5, 2022

(51) Int. Cl. A61F 13/20; A61F 13/34

(52) U.S. Cl. CPC A61F 13/2074; A61F 13/34; A61F 2013/8455

(57) ABSTRACT

A feminine hygiene assembly for absorbing menstrual fluids includes a tampon which is comprised of a fluid absorbent material to absorb menstrual fluids when the tampon is inserted into a vagina. A pair of chambers is each integrated into an interior of the tampon and each of the chambers contains a fluid. A first pull cord is integrated into the tampon and extends outwardly from the tampon for removing the tampon from the vagina. A second pull cord is integrated into the tampon and extends outwardly from the tampon. The second pull cord releases the fluid contained in each of the chambers when the second pull cord is pulled outwardly from the tampon to enhance removing the tampon from the vagina thereby increasing comfort for a user.

### (b) CROSS-REFERENCE TO RELATED APPLICATIONS

Not Applicable

### (C) STATEMENT REGARDING FEDERALLY SPONSORED RESEARCH OR DEVELOPMENT

Not Applicable

### (D) THE NAMES OF THE PARTIES TO A JOINT RESEARCH AGREEMENT

Not Applicable

### (E) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (E) STATEMENT REGARDING PRIOR DISCLOSURES BY THE INVENTOR OR JOINT INVENTOR

Not Applicable

### (G) BACKGROUND OF THE INVENTION

### (1) Field of the Invention

The disclosure relates to feminine hygiene devices and more particularly pertains to a new feminine hygiene device for absorbing menstrual fluids. The device includes a tampon that has a pair of chambers integrated into the tampon which each contains a fluid. The device includes a first pull cord extending away from the tampon for removing the tampon from a vagina. The device includes a second pull cord which extends into each of the chamber and which releases the fluid from the chambers when the second pull cord is pulled to enhance removing the tampon from the vagina.

### (2) DESCRIPTION OF RELATED ART INCLUDING INFORMATION DISCLOSED UNDER 37 CFR 1.97 AND 1.98

The prior art relates to feminine hygiene devices including a tampon device that has a tab attached to a pull string of the tampon to facilitate the pull string to be located by touch. The prior art discloses a variety of tampon devices that each includes a pull string which forms a closed loop. The prior art discloses a tampon device that includes a tampon, a pull string attached to the tampon and a panel attached to the pull string for enhancing gripping the pull string. The prior art discloses a tampon device that comprises an outer tube and an inner absorbent member that is positioned within the outer tube.

### (h) BRIEF SUMMARY OF THE INVENTION

An embodiment of the disclosure meets the needs presented above by generally comprising a tampon which is comprised of a fluid absorbent material to absorb menstrual fluids when the tampon is inserted into a vagina. A pair of chambers is each integrated into an interior of the tampon and each of the chambers contains a fluid. A first pull cord is integrated into the tampon and extends outwardly from the tampon for removing the tampon from the vagina. A second pull cord is integrated into the tampon and extends outwardly from the tampon. The second pull cord releases the fluid contained in each of the chambers when the second pull cord is pulled outwardly from the tampon to enhance removing the tampon from the vagina thereby increasing comfort for a user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (I) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective phantom of a feminine hygiene assembly according to an embodiment of the disclosure.

FIG. 2 is a bottom view of an embodiment of the disclosure.

FIG. 3 is a front view of an embodiment of the disclosure.

FIG. 4 is a cross sectional view taken along line 4-4 of FIG. 3 of an embodiment of the disclosure.

FIG. 5 is a magnified detail view taken from circle 5 of FIG. 4 of an embodiment of the disclosure.

FIG. 6 is a perspective in-use view of an embodiment of the disclosure.

### (J) DETAILED DESCRIPTION OF THE INVENTION

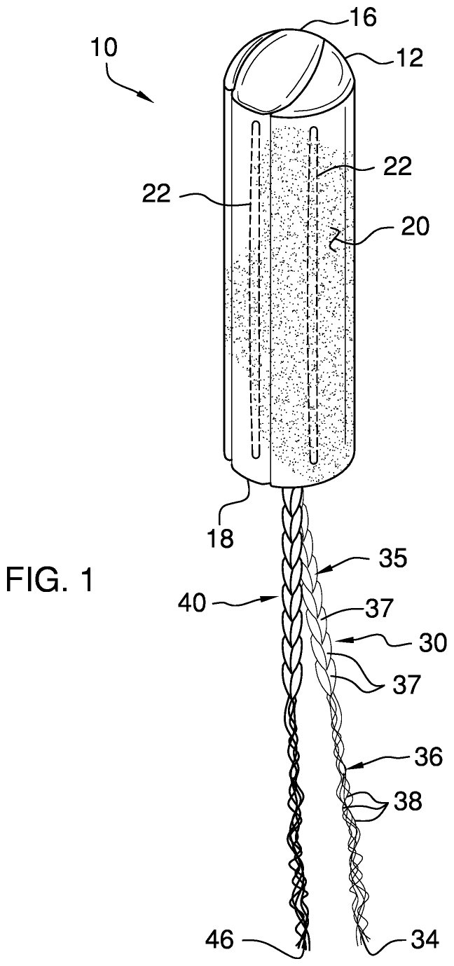

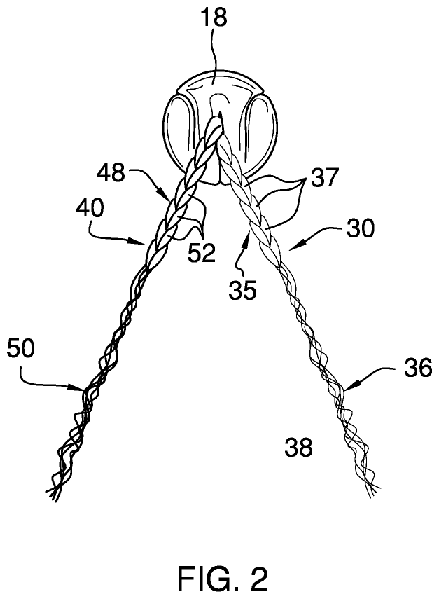

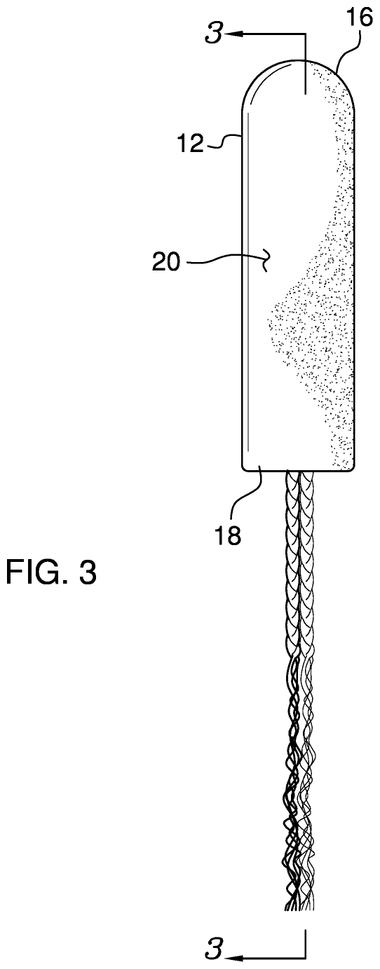

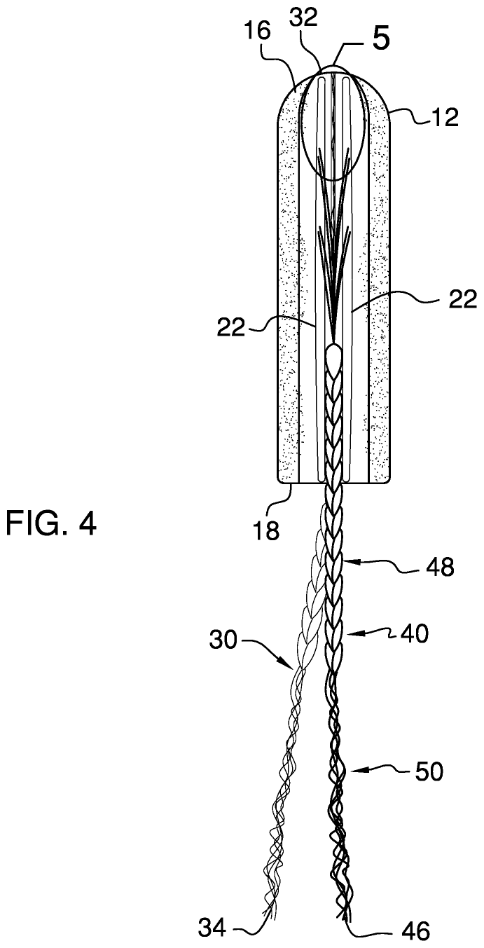

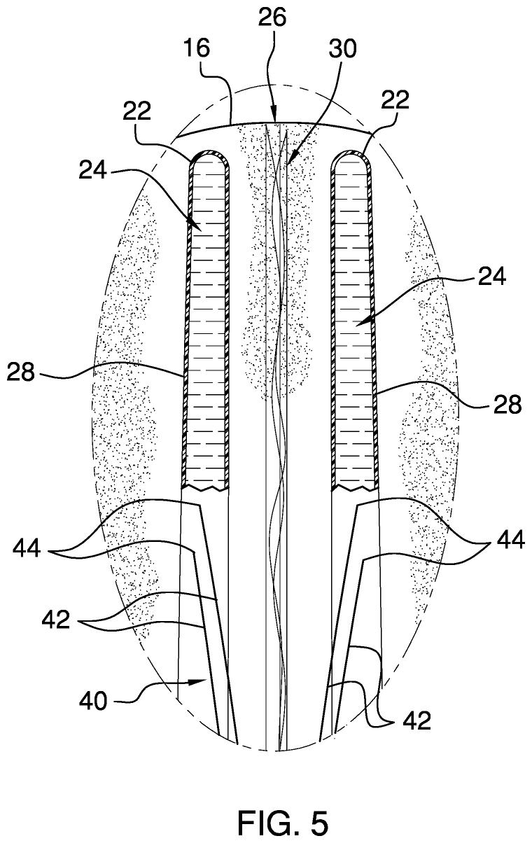

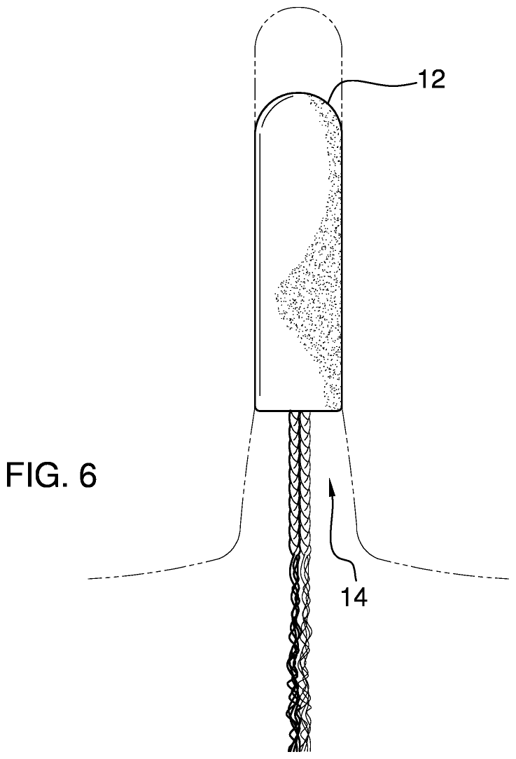

With reference now to the drawings, and in particular to FIGS. 1 through 6 thereof, a new feminine hygiene device embodying the principles and concepts of an embodiment of the disclosure and generally designated by the reference numeral 10 will be described.

As best illustrated in FIGS. 1 through 6, the feminine hygiene assembly 10 generally comprises a tampon 12 that is comprised of a fluid absorbent material to absorb menstrual fluids when the tampon 12 is inserted into a vagina 14. The tampon 12 has a top end 16, a bottom end 18 and an outer surface 20 extending between the top end 16 and the bottom end 18. The top end 16 is rounded, the bottom end 18 is flattened, the outer surface 20 is continuously arcuate about an axis extending between the top end 16 and the bottom end 18 such that the tampon 12 has a cylindrical shape and the tampon 12 is elongated between the bottom end 18 and the top end 16. Furthermore, the tampon 12 may comprise a pad that is folded onto itself along an axis extending between the top end 16 and the bottom end 18 to define the cylindrical shape of the tampon 12.

A pair of chambers 22 is each integrated into an interior of the tampon 12 and each of the chambers 22 contains a fluid 24. Each of the chambers 22 is elongated to extend a substantial distance between the top end 16 and the bottom end 18, and each of the chambers 22 is positioned on opposite sides of a centerline 26 of the tampon 12. The fluid 24 contained in each of the chambers 22 may comprise a saline solution of other type of fluid that is non-toxic to humans. Furthermore, each of the chambers 22 has an outer wall 28 that is comprised of a fluid 24 impermeable material to inhibit the tampon 12 from absorbing the fluid 24 in the chambers 22.

A first pull cord 30 is integrated into the tampon 12 and extends outwardly from the tampon 12 thereby facilitating the first pull cord 30 to be gripped for removing the tampon 12 from the vagina 14. The first pull cord 30 has an upper end 32 and a lower end 34, and the first pull cord 30 extends along the centerline 26 of the tampon 12 such that the first pull cord 30 extends between the pair of chambers 22 having the upper end 32 being positioned proximate the top end 16 of the tampon 12. The first pull cord 30 has a braided portion 35 and a stranded portion 36, and the braided portion 35 comprises a series of braids 37 extending from the bottom end 18 of the tampon 12 toward the lower end 34 of the first pull cord 30. The stranded portion 36 comprises a plurality of unbraided strands 38 extending between the braided portion 35 and the lower end 34. Furthermore, the first pull cord 30 is comprised of a fluid absorbent material thereby facilitating the braided portion 35 of the first pull cord 30 to absorb menstrual fluids thereby inhibiting menstrual fluids from leaking from the vagina 14.

A second pull cord 40 is integrated into the tampon 12 and extends outwardly from the tampon 12 and the second pull cord 40 comprises a plurality of strands 42. Each of the strands 42 of the second pull cord 40 has a topmost end 44 and a lowermost end 46, and the second pull cord 40 extends along the centerline 26 of the tampon 12. Furthermore, each of the strands 42 extends into a respective one of the chambers 22 such that the topmost end 44 of each of the strands 42 is positioned inside of the respective chamber 22. The second pull cord 40 has a braided portion 48 and a stranded portion 50, and the braided portion 48 of the second pull cord 40 comprises a series of braids 52 extending from the bottom end 18 of the tampon 12 toward the lowermost end 46 of the strands 42 of the second pull cord 40.

The stranded portion 50 of the second pull cord 40 comprises the strands 42 of the second pull cord 40 which are unbraided and which extends between the braided portion 48 of the second pull cord 40 and the lowermost end 46 of the strands 42. The second pull cord 40 releases the fluid 24 contained in each of the chambers 22 when the second pull cord 40 is pulled outwardly from the tampon 12. Each of the strands 42 of the second pull cord 40 that extends into the respective chambers 22 causes ruptures in the outer wall 28 of the respective chambers 22 when the second pull cord 40 is pulled outwardly from the tampon 12. Subsequently the tampon 12 absorbs the fluid 24 contained in each of the chambers 22 such that the outer surface 20 of the tampon 12 becomes moistened with the fluid 24. In this way the fluid 24 enhances removing the tampon 12 from the vagina 14 in the event that the outer surface 20 of the tampon 12 has become adhered to the vagina 14 thereby increasing comfort for a user. Furthermore, the second pull cord 40 has a color is visually distinct from a color of the first pull cord 30.

In use, the tampon 12 is inserted into the vagina 14 when the user is menstruating to absorb menstrual fluids. The second pull cord 40 is pulled outwardly from the tampon 12 to release the fluid 24 from the chambers 22 into the tampon 12. In this way the tampon 12 becomes moistened to ensure the outer surface 20 of the tampon 12 does not stick to the vagina 14. The first pull cord 30 is pulled to remove the tampon 12 and the fluid 24 that is released from the chambers 22 facilitates the tampon 12 to slide comfortably out of the vagina 14. In this way comfort for the user is enhanced with respect to removing the tampon 12 during menstruation. Furthermore, the fluid 24 reduces odors that can sometimes occur from removing the tampon 12 thereby enhancing privacy and dignity for the user.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A feminine hygiene assembly comprising:
a tampon being comprised of a fluid absorbent material wherein said tampon is configured to absorb menstrual fluids when said tampon is inserted into a vagina;
a pair of chambers, each of said chambers being integrated into an interior of said tampon, each of said chambers containing a lubricating fluid;
a first pull cord being integrated into said tampon and extending outwardly from said tampon wherein said first cord is configured to be gripped for removing said tampon from the vagina; and
a second pull cord being integrated into said tampon and extending outwardly from said tampon, said second pull cord releasing said lubricating fluid contained in each of said chambers when said second pull cord is pulled outwardly from said tampon thereby facilitating said tampon to absorb said lubricating fluid contained in each of said chambers wherein said lubricating fluid is configured to enhance removing said tampon from the vagina thereby increasing comfort for a user.

2. The assembly according to claim 1, wherein:
said tampon has a top end, a bottom end and an outer surface extending between said top end and said bottom end, said top end being rounded, said bottom end being flattened, said outer surface being continuously arcuate about an axis extending between said top end and said bottom end such that said tampon has a cylindrical shape, said tampon being elongated between said bottom end and said top end; and
each of said chambers is elongated to extend a substantial distance between said top end and said bottom end, each of said chambers being positioned on opposite sides of a centerline of said tampon.

3. The assembly according to claim 2, wherein said first pull cord has an upper end and a lower end, said first pull cord extending along said centerline of said tampon such that said first pull cord extends between said pair of chambers having said upper end being positioned proximate said top end of said tampon.

4. The assembly according to claim 3, wherein:
said first pull cord has a braided portion and a stranded portion;
said braided portion comprises a series of braids extending from said bottom end of said tampon toward said lower end of said first pull cord; and
said stranded portion comprises a plurality of unbraided strands extending between said braided portion and said lower end.

5. The assembly according to claim 4, wherein said first pull cord is comprised of a fluid absorbent material thereby facilitating said braided portion of said first pull cord to absorb menstrual fluids wherein said first pull cord is configured to inhibit menstrual fluids from leaking from the vagina.

6. The assembly according to claim 2, Wherein said second pull cord comprises a plurality of strands, each of said strands of said second pull cord having a topmost end and a lowermost end, said second pull cord extending along said centerline of said tampon, each of said strands extending into a respective one of said chambers such that said topmost end of each of said strands is positioned inside of said respective chamber.

7. The assembly according to claim 6, wherein:
said second pull cord has a braided portion and a stranded portion;
said braided portion of said second pull cord comprises a series of braids extending from said bottom end of said tampon toward said lowermost end of said strands of said second pull cord; and
said stranded portion of said second pull cord comprises said strands of said second pull cord being unbraided extending between said braided portion of said second pull cord and said lowermost end of said strands.

8. The assembly according to claim 1, wherein said second pull cord having a color being visually distinct from a color of said first pull cord.

9. A feminine hygiene comprising:
a tampon being comprised of a fluid absorbent material wherein said tampon is configured to absorb menstrual fluids when said tampon is inserted into a vagina, said tampon having a top end, a bottom end and an outer surface extending between said top end and said bottom end, said top end being rounded, said bottom end being flattened, said outer surface being continuously arcuate about an axis extending between said top end and said bottom end such that said tampon has a cylindrical shape, said tampon being elongated between said bottom end and said top end;
a pair of chambers, each of said chambers being integrated into an interior of said tampon, each of said chambers containing a lubricating fluid, each of said chambers being elongated to extend a substantial distance between said top end and said bottom end, each of said chambers being positioned on opposite sides of a centerline of said tampon;
a first pull cord being integrated into said tampon and extending outwardly from said tampon wherein said first cord is configured to be gripped for removing said tampon from the vagina, said first pull cord having an upper end and a lower end, said first pull cord extending along said centerline of said tampon such that said first pull cord extends between said pair of chambers having said upper end being positioned proximate said top end of said tampon, said first pull cord having a braided portion and a stranded portion, said braided portion comprising a series of braids extending from said bottom end of said tampon toward said lower end of said first pull cord, said stranded portion comprising a plurality of unbraided strands extending between said braided portion and said lower end, said first pull cord being comprised of a fluid absorbent material thereby facilitating said braided portion of said first pull cord to absorb menstrual fluids wherein said first pull cord is configured to inhibit menstrual fluids from leaking from the vagina; and
a second pull cord being integrated into said tampon and extending outwardly from said tampon, said second pull cord comprising a plurality of strands, each of said strands of said second pull cord having a topmost end and a lowermost end, said second pull cord extending along said centerline of said tampon, each of said strands extending into a respective one of said chambers such that said topmost end of each of said strands is positioned inside of said respective chamber, said second pull cord having a braided portion and an stranded portion, said braided portion of said second pull cord comprising a series of braids extending from said bottom end of said tampon toward said lowermost end of said strands of said second pull cord, said stranded portion of said second pull cord comprising said strands of said second pull cord being unbraided extending between said braided portion of said second pull cord and said lowermost end of said strands, said second pull cord releasing said lubricating fluid contained in each of said chambers when said second pull cord is pulled outwardly, from said tampon thereby facilitating said tampon to absorb said lubricating fluid contained in each of said chambers wherein said lubricating fluid is configured to enhance removing said tampon from the vagina thereby increasing comfort for a user, said second pull cord having a color being visually distinct from a color of said first pull cord.
